(19) United States
(12) Patent Application Publication
(10) Pub. No.: US 2006/0178748 A1
(43) Pub. Date: Aug. 10, 2006

(54) Implants and delivery system for treating defects in articulating surfaces

(76) Inventors: Fred B. Dinger III, San Antonio, TX (US); Neil C. Leatherbury, San Antonio, TX (US); Jeffrey S. Wrana, San Antonio, TX (US); John Pak, Colorado Springs, CO (US); Gabriele G. Niederauer, San Antonio, TX (US)

Correspondence Address: GREENLEE WINNER AND SULLIVAN P C, 4875 PEARL EAST CIRCLE, SUITE 200, BOULDER, CO, 80301, US

(21) Appl. No.: 11/290,142

(22) Filed: Nov. 30, 2005

Related U.S. Application Data
(63) Continuation-in-part of application No. 11/052,626, filed on Feb. 7, 2005.
(60) Provisional application No. 60/632,050, filed on Nov. 30, 2004.
(60) Provisional application No. 60/542,640, filed on Feb. 5, 2004.

(51) Int. Cl. A61F 2/30; A61B 17/17

(52) U.S. Cl. 623/18.11; 606/96

(57) ABSTRACT

The invention provides implant plugs having a complex clinically acceptable proximal surface. The invention also provides multi-phase implant plugs which have a nonplanar proximal surface. Suitable implant proximal surface shapes include, but are not limited to, concave surfaces, convex surfaces, faceted domes and angled surfaces formed by the convergence of two facets. The implants of the invention are suitable for repair of tissue defects in articulating surfaces. The invention also provides delivery devices and methods for delivering the implants of the invention. The invention also provides methods for creating defects suitable for use with the implants of the invention.

### CROSS-REFERENCE TO RELATED APPLICATIONS

This application claims the benefit of U.S. Provisional Application 60/632,050, filed Nov. 30, 2004, and is a continuation-in-part of U.S. application Ser. No. 11/052,626, filed Feb. 7, 2005, which claims the benefit of U.S. Provisional Application 60/542,640 filed on Feb. 5, 2004, all of which are incorporated by reference to the extent not inconsistent with the disclosure herein.

### BACKGROUND OF THE INVENTION

This invention relates to implants, devices, and methods for performing repairs of cartilage and bone defects, where the defects are located on nonplanar or complex surfaces.

It is well known in the art that implants can be inserted into damaged bone or cartilage layers to treat injuries to those tissue layers. One type of procedure involves inserting plugs of healthy bone or cartilage that are harvested from a healthy area of the patient's body and transplanted into the defect, as disclosed in U.S. Pat. Nos. 5,152,763 (Johnson et al.), 5,919,196 (Bobic et al.), and 6,358,253 (Torrie et al.). In the alternative an implant can consist of synthetic material, such as porous biocompatible foams or polymers, for example as disclosed in U.S. Pat. Nos. 4,186,448 (Brekke et al.), 5,607,474 (Athanasiou et al.), and 5,716,413 (Walter et al).

Articular cartilage is a tissue that covers the articulating surfaces between bones in joints, such as the knee, elbow or ankle. In the skeletal system, most of the major articulating joints, such as the knee or the hip, are comprised of relatively congruous surfaces which move smoothly through a range of motion. In certain articulating spaces, such as the ankle, the surfaces are comprised of more complicated geometries. For example, in the talus articulating surfaces are found on at least five surfaces. These articulating surfaces often converge in sharp transition points, creating a complicated geometry for surgical treatment in the event of acute or traumatic injury. Current therapies are usually limited to debridement, restricted motion, palliative drug therapy, osteochondral transplantation, or as a last resort, joint fusion. To recapitulate the articulating surface in an effort to reduce pain and restore function, the surgeon has few options. Currently, one common (although unpopular) option is to perform an osteochondral transplant from an articulating surface in the knee to the ankle. It is often difficult if not impossible to match the geometry between the donor and recipient surfaces, often resulting in marginal or unsatisfactory treatment. If the defect or injury is on the medial or lateral ridge of the talus, thus bridging two intersecting articular surfaces, there is no anatomical site from which a satisfactorily congruous donor tissue can be harvested.

A number of patents describe materials, devices and methods suitable for cartilage repair. U.S. Pat. No. 5,716,413 to Walter et al. describes moldable, hand-shapable biodegradable implant materials suitable for cartilage repair. U.S. Pat. No. 5,876,452 to Athanasiou et al. describes biodegradable, porous, polymeric implant materials for cartilage repair. U.S. Pat. No. 6,511,511 to Slivka et al. describes fiber-reinforced, porous, biodegradable implant devices suitable for cartilage repair.

Several patents describe multi-phase materials or devices for cartilage repair. U.S. Pat. No. 5,607,474 to Athanasiou et al. describes a multi-phase bioerodible implant/carrier, including implants having a layer with properties similar to those of cartilage and a layer with properties similar to those of bone. U.S. Pat. No. 6,264,701 to Brekke teaches devices having a first region with an internal three-dimensional architecture to approximate the histologic pattern of a first tissue; and a second region having an internal three-dimensional architecture to approximate the histologic pattern of a second tissue. U.S. Pat. Nos. 6,265,149 to Vyakarnam et al. and 6,454,811 to Sherwood et al. teach use of gradients in composition and/or microstructure and/or mechanical properties. U.S. Pat. Nos. 6,626,945 and 6,632,246 to Simon et al. describe cartilage repair plugs having a composite structure. U.S. Pat. No. 6,626,945 to Simon et al. teaches a variety of cartilage plug configurations, including two plug embodiments having an upper layer joining the plugs in which the upper surface of the upper layer is convex.

Current devices for inserting tissue implants, either bone or cartilage transplants or synthetic materials, are deficient for inserting implants in complex surfaces which are not planar or smoothly curved. U.S. Pat. No. 6,358,253 (Torrie et al.) teaches methods for orienting a guide for use with surgical instruments perpendicular to a curved bone surface. In one configuration, the tissue-engaging portion of the guide is shaped so that a rim is formed above a flange. In use, the flange is seated in the bone and the rim contacts and is flush with the bone completely around its circumference. Torrie et al. also mention a configuration in which the tissue-engaging portion is in the form of an enlarged lip having a slightly concave surface

In implant procedures, defects of variable depths are often presented. In order for the implant, once inserted into the defect, to evenly match the surface of the surrounding tissue without protruding or forming a cavity, the depth of the defect must be determined and the length of the implant tailored to fit the defect. Generally, it is difficult to determine the exact depth of a defect and, therefore, to insert an implant with the correct length.

U.S. Pat. No. 5,782,835 (Hart et al.) teaches a bone plug emplacement tool comprising a cylinder with an internal bore along the longitudinal axis and a stem disposed for co-axial movement within the internal bore. A bone plug placed in the internal bore is delivered into the defect when the stem is advanced through the bore. However, the tool does not provide means for determining the depth of the defect or for tailoring the length of the implant to fit the defect.

U.S. Pat. No. 6,395,011 (Johanson et al.) similarly teaches a device comprising a push rod within a hollow cylinder for harvesting and implanting bone plugs. In addition, the device includes a translucent or transparent tip permitting the surgeon to view the bone plug during implantation. Although this is an improvement in that it allows the length of the bone plug to be determined after harvesting, it also does not provide means to determine the depth of the defect.

There remains a need in the art for improved implants, surgical equipment, and repair methods for defects in tissue having a nonplanar or complex surface.

### SUMMARY OF THE INVENTION

Defects may occur such that the shape of the tissue surface in the defect area is complex. For example, it may be desirable to locate an implant along a ridge between two articulating surfaces.

The present invention provides a plug implant with a complex proximal surface for implantation into a tissue defect. With reference to an implant, the “proximal surface” refers to the surface of the implant which, when inserted in the tissue defect, will be closest to the surface of the surrounding tissue. The proximal surface of the implant is designed to be a clinically acceptable replacement for tissue at the defect site. The proximal surface of the implant is also congruous with the tissue which surrounds the implant once it is implanted.

In an embodiment, the proximal surface of the implant comprises two facets converging to form an angled surface. Such a device can be used to match converging articular surfaces in the talus, typically the talar dome and surfaces which articulate with either the medial or lateral malleolus.

In other embodiments, the proximal surface of the implant can be concave or convex. Another application where an implant with a complex articulating surface can be used to restore anatomical function is in the knee. For example, the implants of the invention can be used in the trochlea, the patella, or the patello-femoral joint. The implant can be constructed with a concave shape to match the trochlear sulcus of the femur. Similarly, the implant can be fabricated with a convex, slightly rounded surface to match the surface of the patella.

Still another example of a complex geometry where an implant with a complex surface would be useful is the small joints of the hands and feet. For example, the carpometacarpal, tarsal joints, and metatarsal joints (including metatarsal head joints) represent complex, highly curved surfaces that require implants with complex geometries.

Other examples of joints suitable for the implants of the present invention include the temporomandibular joint (TMJ) of the jaw bone, spine joints (including vertebra and facet joint), and the hip, shoulder, and elbow.

In an embodiment, the implant is a synthetic implant. The implant may be a single or multi-phase construct. A dual phase implant can be used to simulate a combination of cartilage and bone. A multi-phase implant with three phases could be used to simulate a surface with three adjacent tissues, such as articular cartilage, cancellous bone, and cortical bone. Such an implant could be useful in reconstructing a damaged femoral or tibial epiphysis. In another embodiment, the various layers may be separated by a non-permeable film to isolate the different portions of the multiphase implant construct.

The present invention additionally provides a bone and/or cartilage implant delivery tool, which allows for measuring, sizing, and delivering of the implants of the invention to a bone and/or cartilage defect of known or unknown depth, the defect being located in tissue having a complex surface. The delivery tool may be partially or completely translucent or transparent. The present invention also provides methods for implanting the implants of the invention in a bone or cartilage tissue having a complex surface.

The devices of the invention are suitable for treatment of any bone or cartilage defect that is accessible by the device. Furthermore, the device is suitable for use with bone and cartilage transplants as well as synthetic implants. As used herein, “implant” includes implants made from synthetic materials and implants that are bone and cartilage transplants.

The implant delivery devices of the present invention are related to those described in U.S. patent application Ser. No. 10/785,388, filed Feb. 23, 2004, which is hereby incorporated by reference to the extent not inconsistent with the disclosure herein.

The delivery device of the present invention includes a tubular outer shaft having a proximal and a distal end and an internal bore along the longitudinal axis. With respect to the delivery device, “proximal” refers to the end of the device initially oriented closest to the patient's body and used in measuring the depth of the defect as described below. “Distal” refers to the end of the device initially oriented away from the patient's body and used to contain the implant. The internal bore of the outer shaft is sized to accommodate the diameter of the implant or the profile of the implant if the implant is non-cylindrical. In addition, the proximal and distal ends of the outer shaft are shaped to conform to the shape of the tissue surrounding the defect.

A cylindrical inner shaft, also having proximal and distal ends, is disposed within the internal bore in the outer shaft, wherein the proximal end of the inner shaft is suitable for insertion into a defect. By “suitable for insertion into a defect” it is meant that the proximal end of the inner shaft has a size and shape allowing it to fit within a bone and/or cartilage defect without distorting the defect or damaging the tissue layers. In the present invention, the distal end of the inner shaft has a size and shape similar to the size and shape of the proximal surface of the implant. The shaped surface of the inner shaft helps to keep the implant in proper orientation. The inner shaft has a diameter that also allows it to be slidably engaged with the outer shaft. “Slidably engaged” means the inner shaft can slide within the bore in the outer shaft. The inner shaft may be solid or have a cannula through its center. The inner shaft and outer shaft are of the same effective length. The inner shaft and the outer shaft are of the same effective length when the proximal end of the outer shaft and the flat end of the inner shaft are placed in contact with a flat surface and the shaped distal ends of the inner and outer shafts are aligned. When the proximal ends of the inner and outer shafts are aligned, the shape on the distal ends of the inner and outer shafts match. Because one end of the inner shaft is contoured, the contoured portions of inner shaft may be longer than the center measuring site.

The delivery device comprises means to provide friction-retarded movement of the inner shaft through the outer shaft. The inner shaft may have a “friction member,” which is herein defined as a section of the inner shaft having a diameter large enough to contact the inner surface of the outer shaft and provide a tight fit within the internal bore, whereby the inner shaft is able to slide within the outer shaft when force is applied, but will not slide within the outer shaft when no force is applied. The friction member may be coated with rubber or other materials to provide additional friction. The surfaces of the outer shaft and inner shaft also may be modified to provide friction-retarded movement. For example, a section of the outer shaft's inner surface may contain small beads and a corresponding section of the inner shaft's outer surface may contain small ridges. When the inner shaft is moved through the outer shaft, the small beads on the outer shaft contact the ridges on the inner shaft and provide additional friction. Alternatively, a section on the inner surface of the outer shaft may contain ridges or serrated teeth that engage ridges or serrated teeth disposed on the corresponding section on the outer surface of the inner shaft. When the inner shaft is moved through the outer shaft, the ridges and/or serrated teeth contact each other and movement is restricted. Other means that prevent unwanted movement of the inner shaft through the outer shaft include otherwise texturing the surfaces of the inner shaft and outer shaft, or coating the surfaces of the inner shaft and outer shaft with a viscous liquid.

In addition, the delivery device may be designed to limit rotation of the inner shaft within the outer shaft. For example, one of a key or keyway may be located on the inner shaft, with the other of key or keyway located on the outer shaft. The interlocking of the key and keyway limits or prevents rotation of the inner shaft within the outer shaft.

When the inner shaft is disposed in the outer shaft so that the inner shaft does not protrude from the proximal end of the outer shaft, inserting an implant into the distal end of the outer shaft displaces the inner shaft towards the proximal end causing a portion of the inner shaft to protrude from the proximal end of the outer shaft. Conversely, when an implant is preloaded into the distal end of the outer shaft, the inner shaft is inserted in the proximal end of the outer shaft and advanced toward the distal end of the outer shaft until the distal end of the inner shaft contacts the implant. At this point, the implant will not extend beyond the distal end of the outer shaft and a portion of the inner shaft will protrude from the proximal end of the outer shaft.

With an implant at least partially inserted into the distal end of the outer shaft, the proximal end of the inner shaft is inserted into a defect of unknown depth. When the proximal end of the inner shaft contacts the bottom of the defect, the outer shaft is advanced towards the defect until the proximal end of the outer shaft effectively conforms to the surface of the tissue surrounding the defect. In relation to the outer shaft, this motion distally advances the inner shaft. As a result, the length of the inner shaft that protrudes from the proximal end of the outer shaft equals the depth of the defect. In addition, this motion displaces the implant in the outer shaft and causes a portion of the implant to extend beyond the distal end of the outer shaft.

The protruding end of the implant, i.e., the portion of the implant protruding from the distal end of the outer shaft, can be cut off with a knife or other cutting device. Other cutting devices suitable for use with the invention include scissors, a guillotine, and cutting devices as disclosed in U.S. patent application Ser. No. 10/785,388, filed Feb. 23, 2004. The remaining length of the implant in the distal end of the outer shaft equals the length of the inner shaft that protrudes from the proximal end of the outer shaft, which also equals the depth of the defect. The proximal end of the device is removed from the defect and the distal end of the device containing the implant is placed over the defect. The proximal end of the inner shaft, which is now the end furthest from the patient's body, is advanced towards the distal end of the outer shaft, which is now the end closest to the patient's body, pushing the implant into the defect.

While the device can be constructed of any materials, including, but not limited to, medical grade plastic or metal, it is preferred that plastic is used to prevent scratching the bone or cartilage surface. In a further embodiment, a series of thin concentric slots cut into the outer surface of the outer shaft provide a gripping surface for easier handling of the device.

A further embodiment of this invention includes at least one slot or window in the distal end of the outer shaft of the device for visualizing the implant. The slot or window may be of any shape that allows the implant to be seen while the implant is disposed within the delivery device. The slot or window can also be covered with transparent material.

A further embodiment of this invention includes tapered leaves in the distal end of the outer shaft. Longitudinal slots are cut in the distal end of the outer shaft, creating opposing leaves. The leaves are the sections of the outer shaft between the longitudinal slots. These leaves can be made to taper slightly inward, creating slight compression on the implant to prevent undesired movement of the implant within the device.

A further embodiment of this invention includes a snap-bead feature on the distal end of the outer shaft for attaching items to the device. The snap-bead feature comprises an annular groove around the distal end of the outer shaft. An attachable item has one or more small beads or a rim that fits into this groove. One such attachable item is a temporary cap that fits over the distal end of the outer shaft to prevent accidental removal of the implant from the device.

In a further embodiment of this invention, the implant is delivered to a defect with bioactive fluids, such as blood, blood concentrate or cell suspension. After the implant has been sized and cut to fit the defect, a cap can be placed around the distal end of the outer shaft and bioactive fluids added via a window or slot. Additionally, a centrifuge can be used to load fluids and the delivery device can be made suitable for use in a centrifuge, i.e., structurally able to withstand the forces during centrifugation without leaking or damaging the implant, when loading fluids to the implant.

This invention also includes a kit comprising at least one implant delivery device. The kit may also include an implant and a knife or other cutting device. The kit may comprise several implant delivery devices having different sizes of internal bores and inner shafts in order to accommodate defects and implants of varying sizes. The delivery devices of this kit can be individually color coded according to size.

The invention also provides apparatus, kits and methods for creation of a defect having a selected location, diameter and depth in tissue having a nonplanar or complex surface. The apparatus and methods create defects which are compatible with the plug implants of the invention.

The terms “tube”, “tubular” and “cylindrical” used to describe the implant delivery device and implant capsule loader do not exclude depressions, reliefs, flats or flutes, or limit the shapes to only round cylinders. A tube is a hollow conduit, the cross-sectional area of which need not be circular or uniform along the length of the tube. The cross-sectional area of a tube can be any shape including, but not limited to, elliptical, hexagonal, octagonal, or irregular.

### BRIEF DESCRIPTION OF THE DRAWINGS

FIG. 1A is a perspective view of a dual phase plug implant having two facets at the proximal surface of the implant converging to form an angled surface. FIG. 1B is a side view of the implant in FIG. 1B.

FIG. 2 shows an implant installed at the intersection of a simulated talar dome and a simulated talar surface which articulates with the medial malleolus.

FIGS. 3A-3C show a dual phase implant having a saddle-shaped surface.

FIGS. 4A-4C show a dual phase implant which has opposing sides of different lengths.

FIG. 5 shows a delivery device of the present invention with the inner shaft removed from the outer shaft.

FIG. 6 shows an assembled delivery device.

FIGS. 7A and 7B show a delivery device of the invention in contact with a ridged tissue area.

FIG. 8 shows an implant delivery device of this invention having longitudinal slots and a snap-bead feature on the distal end of the outer shaft with an inner shaft protruding from the proximal end of an outer shaft.

FIG. 9A is an end view of the inner shaft of the implant delivery device of FIG. 5 comprising a cannula. FIG. 9B is a side view of an inner shaft having ridges. FIG. 9C is an expanded view of the circled section of FIG. 9B showing the ridges in greater detail. The cannula in FIGS. 9B and 9C is shown by dotted lines.

FIG. 10A is an end view of the outer shaft of the implant delivery device of FIG. 5. FIG. 10B is a side view of the outer shaft shown in FIG. 10A. FIG. 10C is an expanded cross-sectional view of the circled section of FIG. 10B showing friction beads on the inner surface of the outer shaft.

FIG. 11A is an end view of a modified inner shaft of the implant delivery device of FIG. 5 comprising two alignment ribs. FIG. 11B is a side view of a modified inner shaft. FIG. 11C is an expanded view of the circled section of FIG. 11B showing serrated teeth along the surface of the inner shaft. The cannula in FIGS. 11B and 11C is shown by dotted lines.

FIG. 12A is an end view of a modified outer shaft of the implant delivery device of FIG. 5 comprising alignment slots. FIG. 12B is a side view of a modified outer shaft. FIG. 12C is an expanded cross-sectional view of the circled section of FIG. 12B showing serrated teeth on the inner surface of the outer shaft.

FIG. 13 illustrates a tool kit for creating cylindrical defects in a complex tissue surface which includes a drill guide 305, a drill sleeve 310, and alternate drill bits 315a, 315b.

FIGS. 14A-14E illustrate a method for creation of a cylindrical defect in a complex tissue surface.

### DETAILED DESCRIPTION OF THE INVENTION

The present invention addresses repair of tissue defects where the surface of the tissue in the defect area is nonplanar. The implants of the invention are suitable for a variety of tissue surface shapes, including, but not limited to, concave and convex surfaces. In an embodiment, the tissue surface has the form of a cylindrical section or a spherical section. The implants of the invention can be suitable for repairing defects in articulating surfaces.

In an embodiment, the surface of the tissue in the defect area has a complex surface. In an embodiment, the complex surface comprises an articulating surface. As used herein, a complex surface has a mean curvature which is not constant across the surface. For example, a complex surface is not planar, cylindrical or spherical. Complex surfaces can include, but are not limited to, concave surfaces, convex surfaces (dome-shaped surfaces), saddle-shaped surfaces and other surfaces where, at a given point, the planar curves formed by the intersection of the surface with two orthogonal planes that contain the normal vector to the surface are not uniformly convex or concave, angled surfaces formed by the intersection of two facets, multifaceted domes and multifaceted bowls. In an embodiment, the complex surface has compound radii of curvature, which means that the surface has at least two different (non-infinite) radii of curvature. The implant need not be symmetrical. In an embodiment, the implant has one plane of symmetry.

Saddle-shaped implants can be used to treat depressed and/or groove areas of joints.

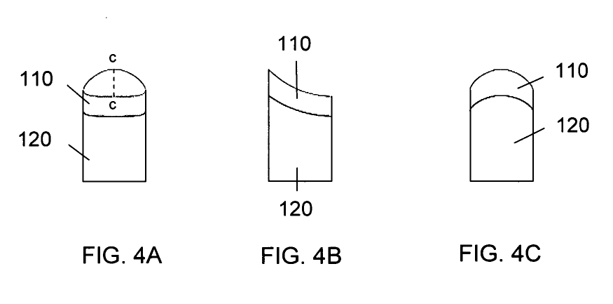

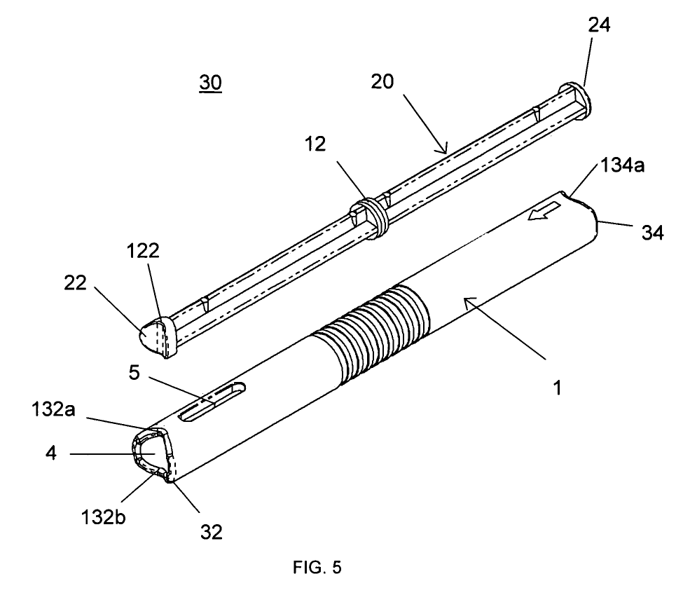

Beveled implants can be used to treat joint ridges. As used herein, in a beveled design the implant is longer on one side than on the opposing side, resulting in a “skijump”-shaped proximal surface which is generally inclined from one side of the implant to the other (as shown in FIG. 4B). In an embodiment, the angle between part of the proximal surface (the surface at the high side of the incline) and the side of the implant is less than ninety degrees. In an embodiment, the angle between the proximal surface at the low side of the incline and the side of the implant can be greater than or equal to 90 degrees. In an embodiment, the angle between the proximal surface at the low side of the incline and the side of the implant is between 90 and 100 degrees. The surface curvature orthogonal to the incline can be zero or non-zero.

Defects suitable for repair by the devices and methods of the present invention include voids in cartilage and/or bone. A defect can be a damaged bone and/or cartilage layer. However, defects are not limited to bone and cartilage injuries. Defects can be intentionally created, such as the hole remaining in bone or cartilage tissue after a plug of healthy bone or cartilage is removed for transplantation. Intentionally created defects also include holes in bone or cartilage tissue created in order to insert autologous or allogenic grafts during ligament or tendon repair surgeries. Holes in the bone or cartilage tissue around a damaged area can also be created to facilitate repair with a plug-shaped implant.

The present invention provides a plug implant with a nonplanar or complex proximal surface for implantation into a tissue defect. The tissue defect is in the form of a void. The plug is sized to have sufficient length to adequately anchor the plug. In an embodiment, the plug is preferably at least about 8 mm long, but certain designs may allow for shorter or longer implants. The proximal surface of the implant provides a clinically acceptable surface shape to replace tissue in the defect area. By clinically acceptable, it is meant that the proximal surface of the implant allows the implanted tissue to function acceptably. For an implant placed in an articulating surface, the shape of the proximal surface of the implant is acceptable if the joint functions acceptably. There may be more than one proximal surface shape which is clinically acceptable for a given defect area. The proximal surface of the implant may be similar, although not necessarily identical, to the surface of the tissue in the defect area before it was damaged. The proximal surface of the implant may also be simpler than the undamaged surface of the tissue in the defect area. The proximal surface of the implant is also designed to be congruous with that of the tissue which surrounds the implanted implant. Congruence of the proximal surface of the implant with the surrounding tissue means that the contour of the perimeter of the proximal surface of the implant is similar, although not necessarily identical, to that of the surrounding tissue.

In an embodiment, the implant is composed of a biomaterial whose proximal surface is shaped to match the contour of a complex or irregular articulating surface. Such a surface can consist of one or more facets articulating through one or more degrees of freedom. For example, in the trochlear sulcus a concave “vee” shape is formed by two facets for the translation of the patella. There is primarily one degree of freedom in the translation, i.e., in a linear direction parallel to the groove of the vee. As another example, in the talus, six articular surfaces translate through at least three degrees of freedom, in the sagital plane, the AP (anterior/posterior) plane, and in rotation.

As used herein, a plug implant is an implant designed to fill a defect hole tightly. A plug implant may be a right or oblique cylinder or a right or oblique prism, or another shape selected to suit the needs of the defect. Ideally, the implant will occupy and maintain the defect area, providing mechanical support to both the surrounding tissues and to the repairing tissues within the defect. In an embodiment, the implants of the invention do not encompass bridged implant designs having a plurality of anchor plugs.

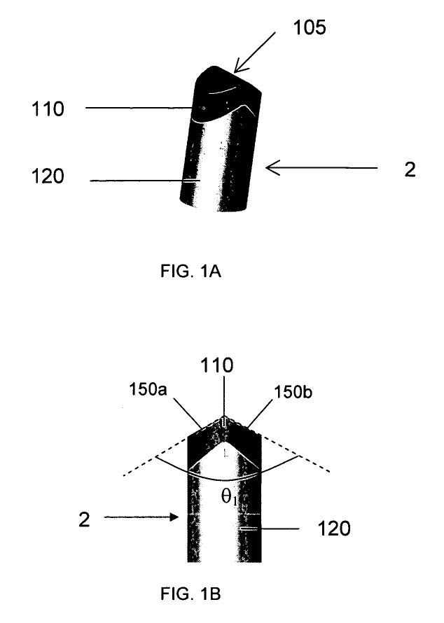

FIGS. 1A and 1B show an exemplary implant of the invention. In FIGS. 1A and 1B, the proximal surface 105 of implant 2 has two facets (150a, 150b in FIG. 1B) converging to form an angled surface. Such an implant can be used to match two converging articular surfaces in the talus, typically the talar dome and the surface of the talus which articulates with either the medial or lateral malleolus. The angle, θ₁, between the two facets depends upon the location of the defect. In different embodiments, the angle between the two facets is between about 70 and about 130 degrees and between about 90 and about 110 degrees. An implant for installation at the intersection of the talar dome and the medial malleolus can have an angle θ₁ of about 100 degrees.

The implant shown in FIGS. 1A and 1B is a dual phase implant which has a proximal layer 110 and a distal layer 120. The proximal layer can be fabricated to match the properties of the cartilage in the defect area and the distal layer formulated to match the properties of the bone in the defect area. This implant would be suitable for filling a defect in both bone and cartilage, so that the distal layer is primarily located in the bone area of the defect and the proximal layer is primarily located in the cartilage area of the defect.

Another example would be an implant to treat defects on the patella. Such an implant could combine concave and convex portions to match the various curvatures on the articulating surface. For example, a defect could extend from the convex ridge of the patella to the concave lateral side, requiring an oblong or elliptical implant with a complex surface. In this case, the delivery the inner diameter of the outer sleeve of the delivery device would match the elliptical profile of the implant, and the proximal and distal ends of the device would match the complex curvature of the patellar surface.

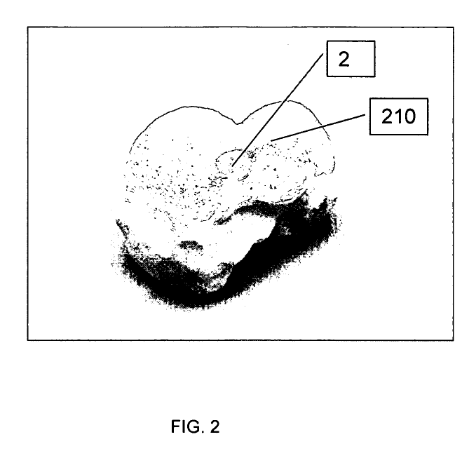

FIG. 2 shows an implant 2 of the invention inserted at the medial ridge 210 of a simulated talus. The implant has two facets.

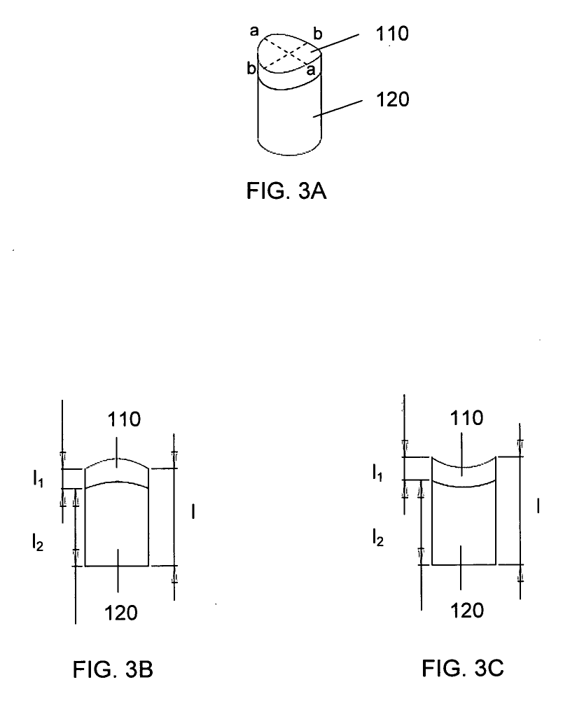

FIGS. 3A-3C show a dual-phase implant of the invention having a saddle-shaped upper surface. FIG. 3A is a perspective view of the invention, while FIG. 3B is a cross-section along b-b and FIG. 3C is a cross-section along a-a. In FIG. 3B and 3C, the total length of the implant, l, the length of the proximal or upper layer, l₁, and the length of the distal or lower layer l₂, are all shown as measured at the edge of the implant. At the edge of the implant, the total length, l, is greater in FIG. 3C than in FIG. 3B. The thickness of the upper layer of the implant is shown as slightly greater in FIG. 3C than in FIG. 3B. In an embodiment, the length, l₁, is between about 15 and about 20 mm and the length of the upper layer, l₁, is between about 3 and about 5 mm.

A cylindrical implant having a saddle-shaped surface can be described by (1) a primary axis of rotation (2) lateral and frontal planes that intersect to form the primary axis and are perpendicular to each other, (3) a transverse plane that is orthogonal to the lateral and frontal planes, (4) a distal flat end that is parallel with the transverse plane and (5) a proximal end that contains a concave surface. In terms of an x-y-z coordinate system where the axis of rotation is coincident with the y axis, the frontal plane is the x-y plane, the lateral plane is the y-z plane, and the transverse plane is the x-z plane. This concave surface is defined by a locus of points described by any mathematical function producing a surface where f(x)=f(−x) on the frontal plane and/or f(z)=f(−z) on the lateral plane and x=0, z=0 is coincident with the axis of symmetry.

FIGS. 4A-4B show another implant of the invention which has a beveled proximal surface. FIGS. 4A and 4C show opposing side views of the implant. FIG. 4B is a cross-section along c-c. In FIG. 4B, the angle of inclination between the proximal surface and the side wall of the implant is smaller at the left side than at the right side. The left side of the implant is also higher than the right side of the implant. The implant shown in FIGS. 4A-4C is suitable for use in the wall of the trochlea.

A cylindrical implant having a beveled surface can be described by (1) a primary axis of rotation (2) lateral and frontal planes that intersect to form the primary axis and are perpendicular to each other, (3) a transverse plane that is orthogonal to the lateral and frontal planes, (4) a distal flat end that is parallel with the transverse plane and (5) a proximal end that contains a concave surface. In terms of an x-y-z coordinate system where the axis of rotation is coincident with the y axis, the frontal plane is the x-y plane, the lateral plane is the y-z plane, and the transverse plane is the x-z plane. This concave surface is defined by a locus of points described by any mathematical function producing a surface where f(x)≢f(−x) on the frontal plane and/or f(z)≢f(−z) on the lateral plane and x=0, z=0 is coincident with the axis of symmetry.

The implant can either be permanent and non-absorbable, bioabsorbable, or bioactive. In an embodiment where the implant is bioabsorbable, as the tissue forms and replaces the native tissue, the implant is slowly absorbed. After an appropriate period of time, the implant is completely absorbed by the body and replaced by functional native tissue. Suitable materials to make these embodiments are known to the art.

In an embodiment, the implant comprises up to four main components: 1) an absorbable polymer, 2) a ceramic, 3) fibers, and 4) a surfactant. The device can be prepared with only the first component; however additional performance properties can be achieved with addition of the other components. Porous materials made with these components can provide a porous polymeric scaffold, incorporate a high level of biologically active or biologically compatible ceramic or mineral, and provide a high level of toughness and strength. When the material includes surfactant, the porous material becomes more wettable, overcoming some of the limitations of the intrinsically hydrophobic material. Table 1 lists typical percentages of each of these four components. Table 2 lists typical physical properties of the formulations in Table 1. In an embodiment, the implant is fabricated from Polygraft™ materials (Osteobiologics, San Antonio, Tex.).

TABLE 1
Exemplary porous material formulations

| Component | Amount (vol %) |
|---|---|
| Polymer | 40-85% |
| Ceramic | 0-40% |
| Fibers | 0-20% |
| Surfactant | 0-5% |

TABLE 2
Physical attributes of the porous material formulations of Table 1:

| | |
|---|---|
| Porosity | 30-90% |
| Average pore size | 10-600 μm |
| Compressive strength | 0.5-30 MPa |
| (parallel to fiber | |
| orientation) | |
| Time for complete | 6 weeks to 2 years |
| degradation | |

In an embodiment, the porosity of the implant is between about 50% and about 90%. In different embodiments, the porosity of the implant is greater than about 50% or greater than about 70%. Preferably, the implant is sufficiently porous to allow for tissue ingrowth. In an embodiment, the average pore size of the implant is between about 10 microns and about 2000 microns, between about 50 microns and about 900 microns and about 100 microns to about 600 microns. The implant can have a layer that has a higher porosity to more closely simulate cartilage and a layer that has a lower porosity to more closely simulate bone. The implant can also have a portion or layer that has a higher porosity to encourage tissue ingrowth and a portion or layer that has lower porosity to increase the mechanical properties. For example, a layer may have a central portion which has a lower porosity (between about zero and about 30%), surrounded by a ring of higher porosity (between about 50% and about 90%). The porous portion of the implant can be capable of soaking up fluids such as blood or bone marrow and therefore can be loaded with bioactive agents, drugs or pharmaceuticals.

Both autologous and bioactive agents can be used with the implants of the invention. Autologous bioactive agents include, but are not limited to, concentrated blood, such as Platelet-Rich Plasma (PRP) and Autologous Growth Factor (AGF), and the patient's own bone marrow.

Synthetic bioactive agents include but are not limited to bone morphogenic proteins (e.g. BMP-2, BMP-7, BMP-12, and BMP-13), growth factors such as platelet derived growth factor (PDGF), fibroblast growth factor (FGF), insulin-like growth factor (IGF), transforming growth factor beta (TGF-β), and other mitogenic or differentiation factors. Other synthetic bioactive agents could be small peptide analogues of the above mentioned or other growth factors. Still other agents could be drugs or pharmacologically active substances which stimulate the growth or differentiation of tissue.

For multi-phase implants, each phase can be loaded with a different bioactive agent for selectively inducing tissue growth into the desired part of the implant. The bioactive agent or agents could be added at the time of surgery, pre-loaded on the implant, or some combination thereof. For example, the bone phase of a dual phase implant could be pre-loaded with a bone stimulating pharmaceutical and the implant provided sterile to the surgeon. At the time of surgery, the surgeon could add a sterile solution of a cartilage-specific growth factor, resulting in an implant with tissue-specific biological activity. A single agent could also be used in multiple phases of an implant.

The implant may also be seeded with cells of the type whose ingrowth is desired. The implant material of this invention can also be preseeded with autologous or allogenic tissue. The autologous or allogenic tissue may be minced or particulated. In an embodiment, the tissue is dermal tissue, cartilage, ligament, tendon, or bone. These allogenic tissues can be processed to preserve their biological structures and compositions, but to remove cells which may cause an immune response. Similarly, autologous tissues can be utilized and processed as described for allografts.

The absorbable polymer forms the core component of porous implants and is needed for formation of the porous structure of the implant material. The polymer selected is soluble or at least swellable in a solvent and is able to degrade in-vivo without producing toxic side products. Biodegradable polymers known in the art are useful in this invention. Typical polymers are selected from the family of poly-lactide, poly-glycolide, poly-caprolactone, poly-dioxanone, poly-trimethylene carbonate, and their co-polymers; however any absorbable polymer can be used. Polymers known to the art for producing biodegradable implant materials include alpha poly hydroxy acids, polyglycolide (PGA), copolymers of glycolide such as glycolide/L-lactide copolymers (PGA/PLLA), glycolide/trimethylene carbonate copolymers (PGA/TMC); polylactides (PLA), stereocopolymers of PLA such as poly-L-lactide (PLLA), Poly-DL-lactide (PDLLA), L-lactide/DL-lactide copolymers; copolymers of PLA such as lactide/tetramethylglycolide copolymers, lactide/trimethylene carbonate copolymers, lactide/δ-valerolactone copolymers, lactide ε-caprolactone copolymers, polydepsipeptides, PLA/polyethylene oxide copolymers, unsymmetrically 3,6-substituted poly-1,4-dioxane-2,5-diones; polyhydroxyalkanate polymers including poly-beta-hydroxybutyrate (PHBA), PHBA/beta-hydroxyvalerate copolymers (PHBA/HVA), and poly-beta-hydroxypropionate (PHPA), poly-p-dioxanone (PDS), poly-δ-valerolatone, poly-ε-caprolactone, methylmethacrylate-N-vinyl pyrrolidone copolymers, polyesteramides, polyesters of oxalic acid, polydihydropyrans, polyalkyl-2-cyanoacrylates, polyurethanes (PU), polyvinyl alcohol (PVA), polypeptides, poly-beta-maleic acid (PMLA), poly(trimethylene carbonate), poly(ethylene oxide ) (PEO), poly(β-hydroxyvalerate) (PHVA), poly(ortho esters), tyrosine-derived polycarbonates, and poly-beta-alkanoic acids. However any absorbable polymer or combination of absorbable polymers, including co-polymers, can be used. The polymer has a molecular weight sufficient to form a viscous solution when dissolved in a volatile solvent, and ideally precipitates to form a soft gel upon addition of a non-solvent. The polymer can be selected as is known to the art to have a desired degradation period. For an implant of this invention, the degradation period is preferably up to about 2 years, or between about 3 weeks and about 1 year, or between about 6 weeks and about 9 months.

The implant can also contain a ceramic component suitable for buffering as detailed in U.S. Pat. No. 5,741,329, to achieve bimodal degradation as detailed in U.S. patent application Ser. No. 09/702,966, or to obtain increased mechanical properties as detailed in U.S. Pat. No. 6,344,496. The implant can include calcium sulfate, tricalcium phosphate or other ceramic to increase mechanical properties. The ceramic component of the device can add both mechanical reinforcement and biological activity to the material. The ceramic (or mineral) component is preferably chosen from calcium sulfate (hemi- or di-hydrate form), salts of calcium phosphate such as tricalcium phosphate or hydroxyapatite, various compositions of Bioglass®, and blends or combinations of these materials. Particles can range in size from sub-micron to up to 1 mm, depending on the desired role of the component chosen. For example, a highly-reinforced composite material can be prepared by incorporating nano-particles of hydroxyapatite. Alternatively, large particles of calcium sulfate (>100 μm) can be incorporated which will dissolve in 4 to 6 weeks, increasing the overall porosity of the material and stimulating bone formation. Incorporation of calcium-containing minerals can also help buffer the degradation of biodegradable polymers to avoid acidic breakdown products.

Addition of fibers to the composite can increase both the toughness and strength of the material, as is well known to the art. The implant can be composed of a fiber-reinforced matrix as detailed in U.S. Pat. No. 6,511,511 and relevant Continuation-In-Part Applications (U.S. Pat. No. 6,783,712 and U.S. application Ser. No. 10/931,474). The fiber and matrix combination is preferably selected such that the mechanical properties of the composite scaffold are tailored to optimal performance. Fibers suitable for use with the invention include both absorbable and nonabsorbable fibers. In an embodiment, the fibers are randomly aligned. In another embodiment, the fibers are preferentially aligned. Preferential alignment of fibers parallel to one another in a porous material can produce anisotropic behavior as described in U.S. Pat. No. 6,511,511, where the strength is increased when the load is applied parallel to the primary orientation of the fibers. In the present invention, up to 30% by mass of the material can be comprised of fibers. Preferred polymeric fiber materials can be selected from the family of poly-lactide, poly-glycolide, poly-caprolactone, poly-dioxanone, poly-trimethylene carbonate, and their co-polymers; however any absorbable polymer could be used. Polysaccharide-based fibers can be chosen from cellulose, chitosan, dextran, and others, either functionalized or not. Non-polymeric fibers can be selected from spun glass fibers (e.g. Bioglass®, calcium phosphate glass, soda glass) or other ceramic materials, carbon fibers, and metal fibers.

The implant may also include a surfactant (˜1% by weight) to further enhance the tissue ingrowth and biocompatibility of the material. Since a majority of the biodegradable polymers are inherently hydrophobic, fluids do not easily absorb and penetrate. The optional addition of a bio-compatible surfactant can improve the surface wettability of the porous construct. This can improve the ability of blood, body fluids, and cells to penetrate large distances into the center of an implant by increasing the capillary action. Examples of bio-compatible surfactants are poly-ethylene oxides (PEO's), poly-propylene oxides (PPO's), block copolymers of PEO and PPO (such as Pluronic surfactants by BASF), polyalkoxanoates, saccharide esters such as sorbitan monooleate, polysaccharide esters, free fatty acids, and fatty acid esters and salts. Other surfactants known to those skilled in the art may also be used. A surfactant incorporated into the polymer at the time of manufacture so that no post-processing is required has no appreciable effect on the manufacturing operation or the creation of the porous structure.

In an embodiment, the implant has a multiple phase structure. In this embodiment, the implant has two or more phases. A multi-phase implant with more than two phases could be used to simulate a surface with three adjacent tissues, such as articular cartilage, cancellous bone, and cortical bone.

In an embodiment, the implant has a dual-phase structure. The two phases may differ in composition, porosity/morphology, mechanical properties, or a combination of these factors. The dual phase structure may be arranged so that the implant has a proximal layer and a distal layer. With reference to an implant layer, “proximal” refers to the layer of the implant which, when inserted in the tissue, will be closest to the surface of the surrounding tissue. In an embodiment, the proximal layer of the implant is formulated to simulate the properties of cartilage, while the distal layer of the implant is formulated to simulate the properties of the bone. Bone generally presents a less porous and stiffer material than overlying cartilage. In this embodiment, the proximal layer of the implant has mechanical properties similar to that of cartilage, with a stiffness (compressive modulus) between about 2 MPa and about 30 MPa and a strength at yield between about 0.5 MPa and about 5 MPa. In addition, the proximal layer of the implant has a higher porosity than the distal layer, between about 70% and about 90%. Furthermore, the proximal layer of the implant is preferably formulated without a bone inductive ceramic component. The distal layer of the implant has mechanical properties similar to that of bone, with a stiffness between about 40 MPa and about 250 MPa and a stress at yield between about 2 MPa and about 20 MPa. The distal layer of the implant has a porosity between about 60% and about 90%. The thickness of the proximal layer of the implant is selected to be approximately the same as that of the cartilage thickness in the desired implant location. In an embodiment, the thickness range of the proximal layer is between about 0.5 and about 2.5 mm, more preferably 1.0 to 1.5 mm for talar dome applications (K. A. Athanasiou, G. G. Niederauer and R. C. Schenck “Biomechanical Topography of Human Ankle Cartilage” Annals of Biomedical Engineering 23 (697-704), 1995).

Any porous portions of the implant can be fabricated through polymer precipitation and vacuum expansion. Methods for the preparation of precipitated polymers are well-known to the art. In general, the process comprises mixing a dried polymer mix with a solvent, e.g. acetone, precipitating the polymer mass from solution with a non-solvent, e.g. ethanol, methanol, ether or water, extracting solvent and precipitating agent from the mass until it is a coherent mass which can be pressed into a mold or extruded into a mold, and curing the composition to the desired shape and stiffness. The optional surfactant is incorporated into the matrix of the material at the time of manufacture. Methods for incorporating reinforcement materials such as fibers and ceramics are known to the art. Methods for incorporating fiber reinforcements, for example, are described in U.S. Pat. No. 6,511,511, hereby incorporated by reference. Kneading and rolling may be performed as described in U.S. Pat. Nos. 6,511,511 and 6,203,573, hereby incorporated by reference. Curing and foaming the polymer in the mold to form a porous implant may then be done.

The complex surface of the implant may be formed by thermal shaping of the surface. During the thermal shaping process, the temperature should be kept sufficiently low so that the pore structure does not collapse. The maximum temperature for thermal shaping depends on the polymer system. For implants having a polylactide-co-glycolide copolymer matrix, a suitable temperature for thermal shaping is between about 140° F. (60° C.) and about 250° F. (121° C.). The complex surface can also be formed by molding, by machining, or by any other suitable means known to those skilled in the art.

A multi-phase implant may be made in a variety of ways. For example, a dual phase implant may be made by forming the proximal and distal layers of the implant separately and then assembling them using solvent and a small amount of dissolved polymer. A dual-phase implant may also be made by forming one layer and then placing that layer in a mold and forming the other layer as described in U.S. Pat. No. 5,607,474. In addition, a dual-phase implant may be made by forming both layers simultaneously in a mold.

In an embodiment, a delivery device suitable for use with the implants of the invention comprises:

a tubular outer shaft having a proximal and distal end, a longitudinal axis, and an internal bore along the longitudinal axis of said outer shaft;

an inner shaft having a distal end and a proximal end suitable for insertion into a defect, said inner shaft adapted to fit within said internal bore of the outer shaft so that the inner shaft and the outer shaft are slidably engaged;

wherein the proximal and distal ends of the outer shaft conform to the surface of the tissue at the perimeter of the defect, and the distal end of the inner shaft conforms to the proximal surface of the implant.

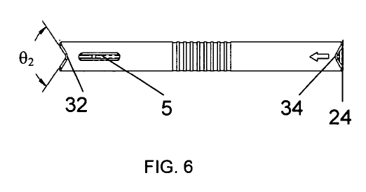

FIGS. 5 and 6 show one embodiment of the implant delivery device 30 of the present invention. To show details of the inner shaft 20, FIG. 5 shows the inner shaft 20 removed from outer shaft 1. In a preferred embodiment, the delivery device 30 has a length suitable for arthroscopic use, i.e., approximately five inches (12.7 cm) to about eight inches (20 cm). The implant delivery device 30 includes a hollow tubular outer shaft 1 having an internal bore 4 along the longitudinal axis. The internal bore 4 extends the entire length of the outer shaft 1 from the distal end 32 to the proximal end 34. Both the distal and proximal ends of the delivery device are shaped to correspond to the shape of the tissue at the perimeter of the defect area. In FIG. 5, the proximal and distal ends of the delivery device each have two indentations or notches (distal notches 132a and 132b and proximal notch 134a are shown in FIG. 5). In FIG. 5, the separation between the centers of the two notches at a given end of the outer tube is 180 degrees. FIG. 6 illustrates the angle, θ₂, of one notch at the distal end of the outer tube. The angles at the distal and proximal ends of the outer tube are the same, as both ends of the outer sleeve of the delivery device will be placed in contact with the complex shape of the tissue surface. The delivery device shown in FIGS. 5 and 6 is suitable for delivery of an implant to a defect located on a ridge. For example, if the defect area is on the medial ridge of the talus, θ₂ can be about 110 degrees. The distal end 32 of the outer shaft 1 can have one or more slots 5 through the outer shaft 1 for visualizing the implant (not shown in FIG. 5) when the implant is in the delivery device 30. Slots 5 can be any shape that allows the implant to be visualized while disposed in the delivery device 30 and can be covered with transparent material.

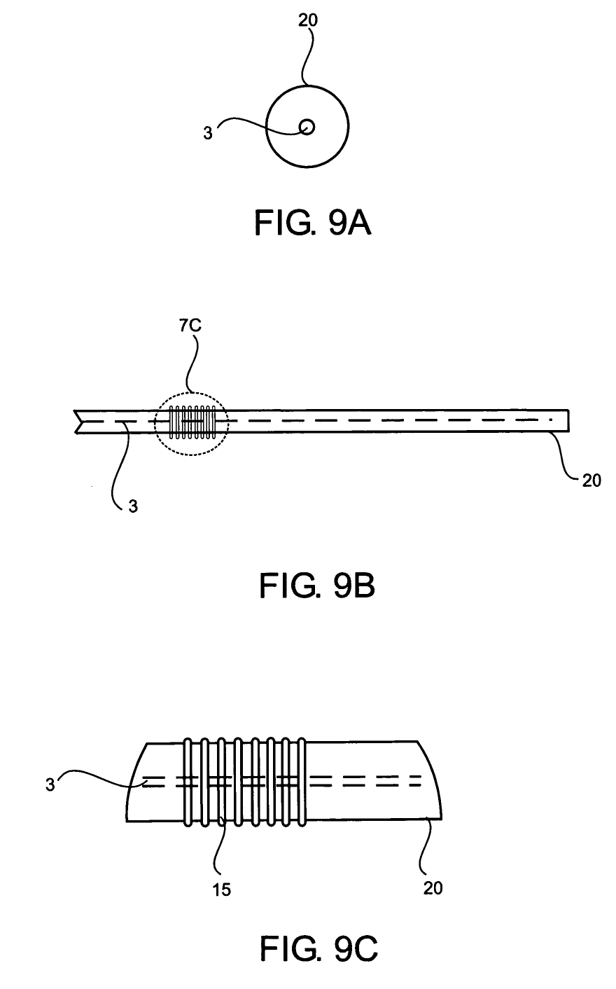

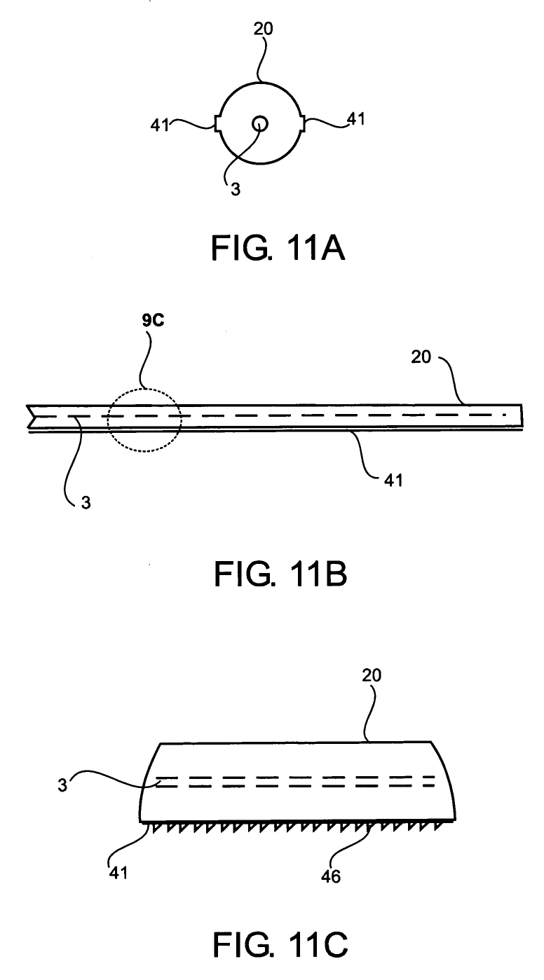

The delivery device 30 illustrated in FIG. 5 further comprises an inner shaft 20 also having distal and proximal ends (22 and 24, respectively). In use, the inner shaft 20 is situated within the outer shaft 1, as shown in FIG. 6 and is able to move proximally and distally through the internal bore 4. The distal end of the inner shaft is shaped to correspond to the proximal surface of the implant. In FIG. 5, the distal end of the inner shaft has a notch 122 (For the delivery device in FIG. 5, the notch angle for the inner shaft is the same as the angle between the implant facets). As shown in FIG. 5, the inner shaft 20 has a friction member 12 which contacts the inner surface of the outer shaft 1. Optionally, the inner shaft may contain a small cannula 3 through its center, as illustrated in FIGS. 9A and 11A. A guide wire attached to the defect by a means such as suturing may be threaded through the cannula.

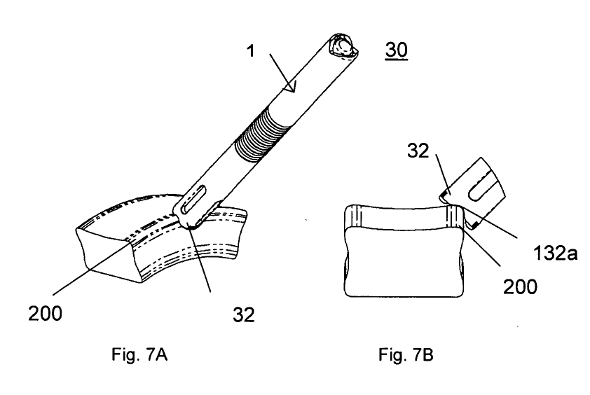

FIGS. 7A and 7B show a delivery device 30 in contact with ridged tissue area 200. Notches 132a and 132b in the distal end of the outer shaft 1 contact the tissue ridge.

The distal and proximal ends of the delivery device may be shaped differently than shown in FIG. 5. For example, for an implant with a concave shape, the implant delivery device would have convex proximal and distal ends for matching the anatomical geometry of the articular surface.

In use, the outer shaft is oriented with respect to the tissue so that the proximal or distal end of the outer shaft effectively conforms to the surface of the tissue surrounding the defect. Since the proximal and distal ends of the outer shaft have been shaped to correspond to the shape of the tissue at the perimeter of the defect area, the outer shaft is oriented to maximize contact between the proximal or distal end of the outer shaft and the tissue surrounding the defect. For example, if the proximal end of the outer shaft is notched and the tissue surrounding the defect is part of a ridge, the outer shaft is oriented such that the notch is placed over the ridge (as illustrated in FIGS. 7A and 7B).

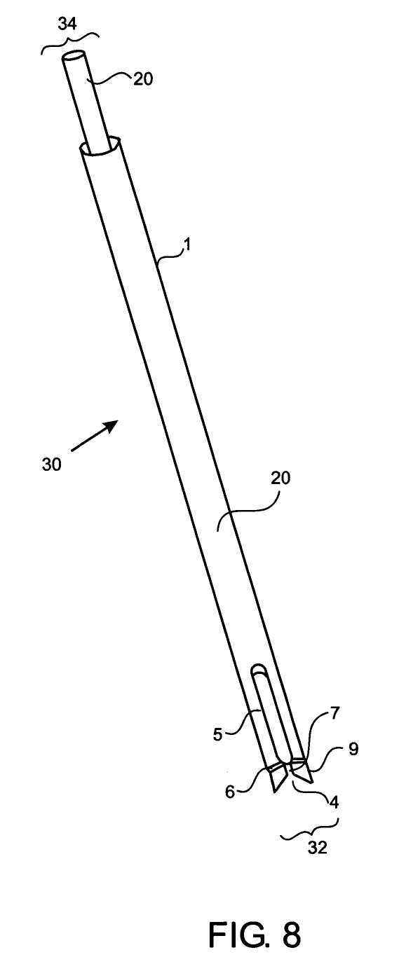

FIG. 8 shows another embodiment of the present invention where the distal end 32 of the delivery device 30 has a small groove 6 running around the outside of the outer shaft 1. In this embodiment, items can attach to the distal end 32 of the outer shaft 1 by having a diameter slightly larger than the outer diameter of the outer shaft 1, fitting over the distal end 32 of the outer shaft 1, and having one or more beads or a rim that snap into the groove 6, thus securing the position of the attached item.

FIG. 8 also shows the delivery device 30 having thin longitudinal slits 7 cut through the distal end 32 of the outer shaft 1 creating leaves 9. Leaves 9 are the sections of the outer shaft 1 between the longitudinal slits 7. The leaves 9 can be made so that they taper slightly inward creating slight compression on the implant (not shown) while in the device 30.

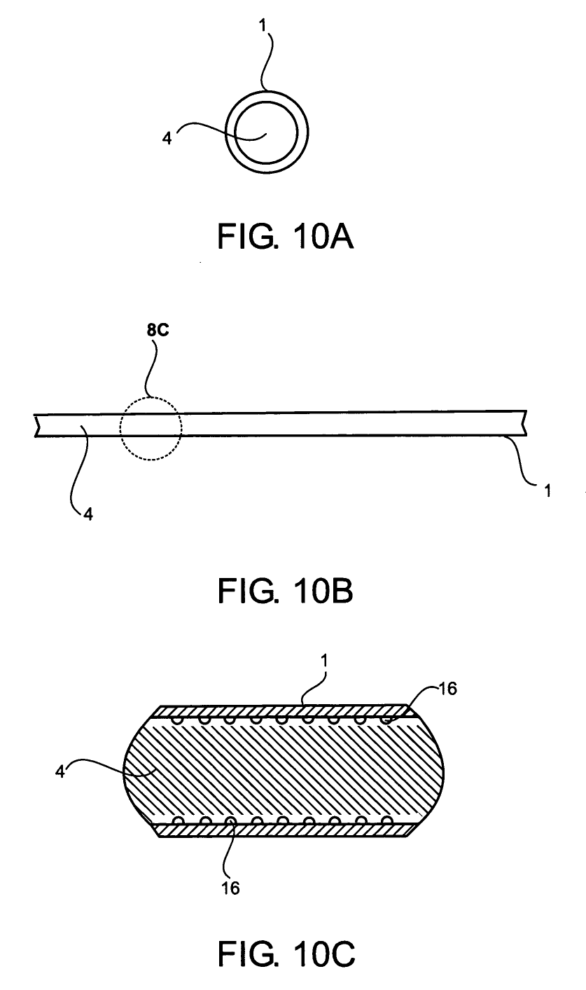

FIGS. 9A-9C show an embodiment of this invention wherein a section of inner shaft 20 comprises ridges 15. Ridges 15 are raised rings around a portion of the outer surface of inner shaft 20. In this embodiment, friction beads 16 are also disposed on the corresponding section of the inner surface of outer shaft 1, as shown in FIG. 10C. The friction beads 16 are raised higher than the surrounding inner surface of outer shaft 1. During proximal and distal movement of inner shaft 20 through internal bore 4 of outer shaft 1, friction beads 16 engage with ridges 15 requiring extra force to continue to advance the inner shaft 20 through the internal bore 4. By “engage with” it is meant that friction beads 16 or serrated teeth 45, as described below, on the inner surface of the outer shaft 1 come into physical contact with ridges 15 or serrated teeth 46, as described below, on the inner shaft 20 providing extra resistance against movement of inner shaft 20 through the internal bore 4.

FIGS. 11A-11C show another embodiment of this invention wherein the outer surface of inner shaft 20 contains at least one alignment rib 41 along the length of inner shaft 20. As shown in FIG. 11A, an alignment rib 41 is a section of the outer surface of inner shaft 20 raised higher than the surrounding surface. Serrated teeth 46 extend out from a section of the alignment rib 41, as shown in FIG. 11C.

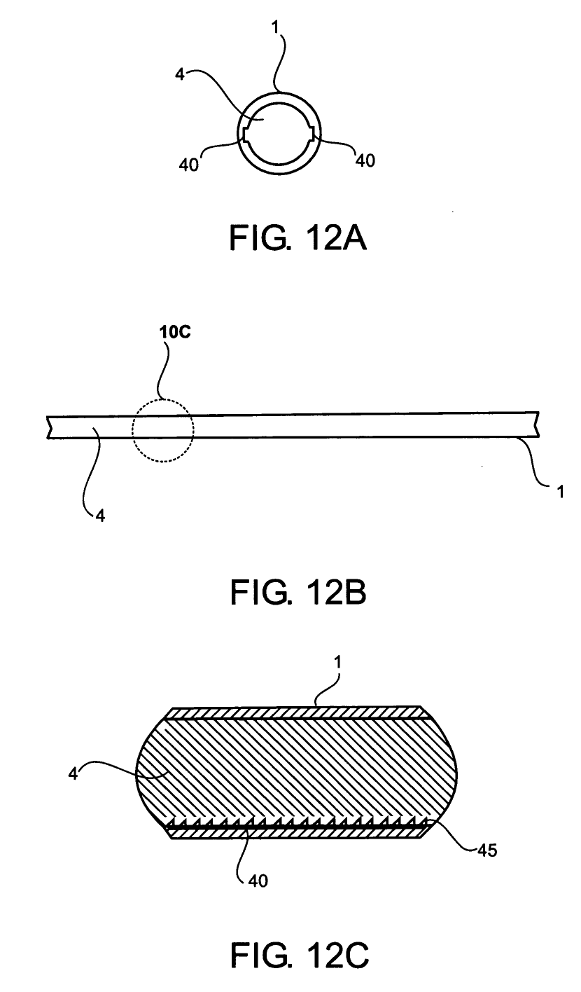

Also in this embodiment, as shown in FIGS. 12A-12C, the outer shaft 1 has at least one alignment slot 40 cut into its inner surface. The depth, position, and number of alignment slots 40 correspond to the height, position, and number of alignment ribs 41 on inner shaft 20 so that the alignment ribs 41 of inner shaft 20 fit into the alignment slots 40 of the inner surface of outer shaft 1. Serrated teeth 45 extend out from a section of alignment slots 40. The section of alignment slot 40 that contains the serrated teeth 45 corresponds to the section of the alignment rib 41 that contains serrated teeth 46, as shown in FIG. 12C.

In this embodiment, inner shaft 20 fits in the internal bore 4 of the outer shaft 1 when alignment rib 41 is aligned with alignment slot 40. During proximal and distal movement of inner shaft 20 through internal bore 4 of outer shaft 1, the serrated teeth 46 along alignment rib 41 contact and engage with serrated teeth 45 along alignment slot 40 preventing unwanted movement.

In this embodiment, the inner shaft fits in the internal bore of the outer shaft when the alignment rib is aligned with the alignment slot. The alignment rib acts as a key and the alignment slot as a keyway. The engagement of the key within the keyway limits or prevents rotation of the inner shaft within the outer shaft. In addition, during proximal and distal movement of the inner shaft through the internal bore of the outer shaft, serrated teeth along the alignment rib can contact and engage with the serrated teeth along the alignment slot preventing unwanted movement. The alignment rib and slot are not required to have serrated teeth for prevention of rotational movement.

Configurations other than a key and keyway can act to limit rotation of the inner shaft within the outer shaft. As a simple example, rotation of the inner shaft within the outer shaft can limited if both have square or rectangular cross-sections and the inner shaft fits closely within the outer shaft.

The invention also provides methods for creating defects in complex tissue surfaces. The tissue surfaces created are suitable for use with the implants and implant delivery devices of the invention. The defect may be created around a tissue injury. In an embodiment, the defects are cylindrical, having a circular cross-section.

In one embodiment, the methods of the invention rely on a drill guide having a proximal end shaped to conform to the shape of the tissue at the perimeter of the defect. The drill guide is in the form of a rigid tube with an interior bore. In use, a surgical instrument such as a drill sleeve is placed into the interior bore of the drill guide. The shaped end of the drill guide stabilizes the position of the instrument. In this embodiment, the proximal end of the drill guide is blunt, rather than sharp. As used herein, the proximal end of the drill guide is the end placed in contact with the tissue.

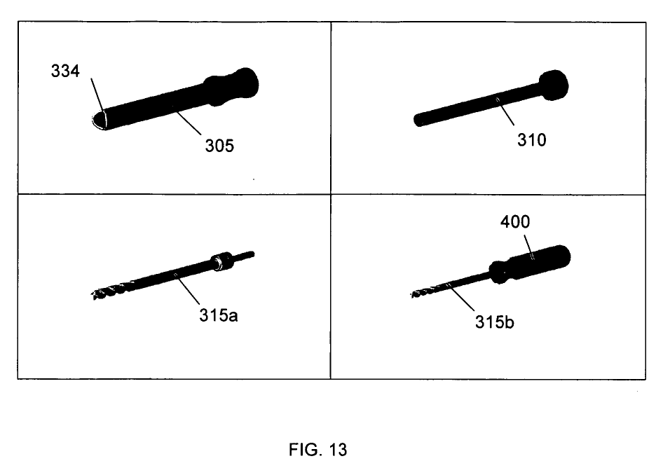

FIG. 13 illustrates a tool kit for creating cylindrical defects in a complex tissue surface which includes a drill guide 305, a drill sleeve 310, and alternate drill bits 315a, 315b. The drill guide is a rigid tube whose proximal end is shaped to allow creation of the defect at a selected angle to the surface. In an embodiment, the defect is created perpendicular to the surface. In the embodiment shown in FIG. 13, the proximal end 334 of drill guide has two notches which enable it to be seated over a tissue ridge. In an embodiment, the drill guide is suitable for placement over the talar medial or lateral ridge and the notch angle is about 110 degrees.

The drill sleeve 310 is a rigid tube with a sharp proximal end. The proximal end of the drill sleeve is placed in contact with the tissue in the defect area and then seated into the tissue. The drill sleeve may be seated in place by any means known to the art, including a mallet. The drill sleeve punches a clean hole through the tissue. The drill sleeve is sized so that the drill guide can guide the position of the drill sleeve and so the drill sleeve can slide within the drill guide. The drill sleeve is also sized to produce the desired defect diameter. In an embodiment, the diameter of the drill sleeve is approximately equal to the desired defect diameter.

The drill bit drills the tissue confined within the drill sleeve to the desired defect depth. The drill bit is sized to remove the tissue confined within the drill sleeve; the tissue is captured in the flutes of the drill. Drilling may be achieved by attaching the drill bit 315a to a standard operating room power drill or by attaching drill bit 315b to a handle 400 as shown.

In an embodiment, the invention provides a method for creating a defect having a selected location, diameter and depth in a tissue having a complex surface, the method comprising the steps of:

a) placing a drill guide so that the proximal end of the guide is in contact with the tissue at the perimeter of the selected defect location, wherein the proximal end of the drill guide is shaped to conform to the shape of the tissue at the perimeter of the selected defect location;

b) inserting a drill sleeve into the interior of the drill sleeve;

c) seating the drill sleeve into the tissue to the selected defect depth;

d) inserting a drill bit into the drill sleeve; and

e) drilling the drill bit to the selected defect depth.

After step e), the drill bit, drill sleeve, and drill guide are removed from the tissue.

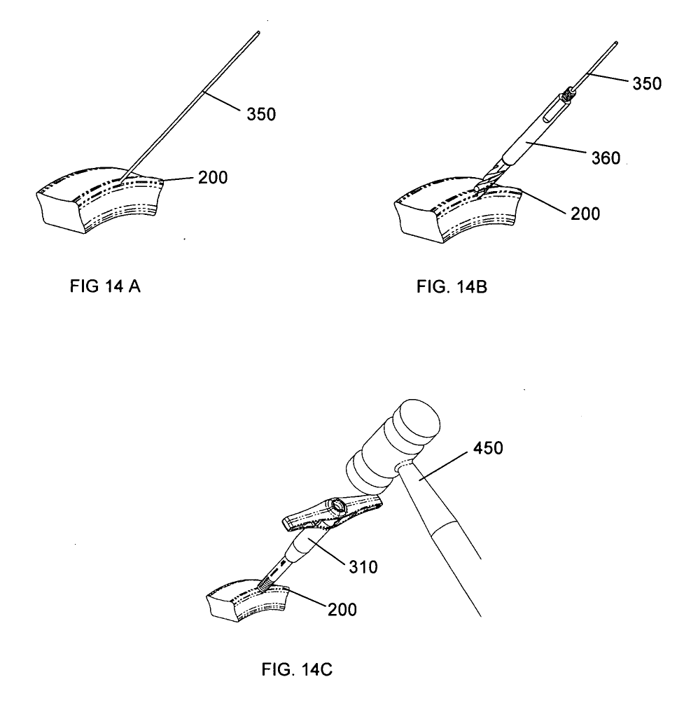

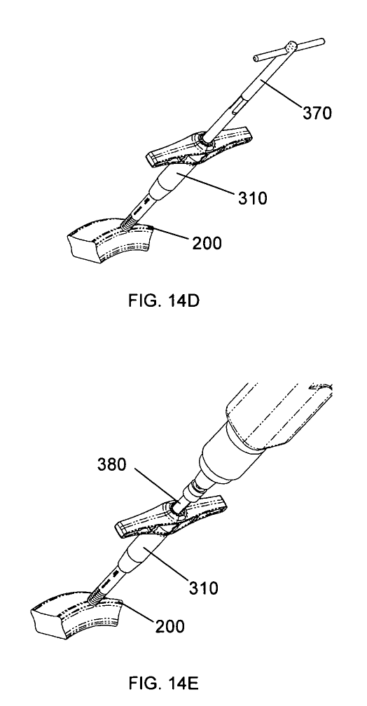

Alternately, the drill guide could be used to guide a punch rather than the drill sleeve. Like the drill sleeve, the punch is a rigid tube with a sharp proximal end. However, the punch is operated so that the tissue inside the punch breaks off near the end of the punch and can be removed with the punch. The tissue inside the punch can be caused to break by twisting or toggling the punch within the drill guide Defects can also be created in a complex surface in other ways. FIGS. 14A-14E illustrate another method for creation of a cylindrical defect which employs a guide wire. The guide wire can make the procedure more stationary and thereby improve the alignment of the defect created. Typically the defect will be created perpendicular to the surface. However in some locations, for example in the hip, it may be desirable to create a defect at an angle other than 90 degrees to the surface. As shown in FIG. 14A, a guide wire 350 is placed in the middle of the selected defect location on tissue ridge 200. One end of the guide wire is seated deeper than the desired depth of the defect to be created. The guide wire may be aligned perpendicular to the surface where the defect is located by being fed through a positioning system (not shown) which is balanced on the surface surrounding the defect. The positioning system may be balanced on three or more legs. As shown in FIG. 14B, a cannulated drill bit is then inserted over the guide wire. The cannulated drill bit is then drilled to the selected defect depth. At this point, the guide wire may be removed or kept in place. As shown in FIG. 14C, a drill sleeve 310 is placed over the cannulated drill bit so that the proximal end of the sleeve is in contact with the tissue and then seated to the desired defect depth with mallet 450. As shown in FIG. 14D, the cannulated drill bit is then removed from the distal end of the drill sleeve using a drill extractor 370. As shown in FIG. 14E, a finishing drill bit 380 is inserted into the drill sleeve and drilled to the selected defect depth. If the guide wire is still in place, the finishing drill is cannulated. The finishing drill removes the remaining tissue, which is captured in the flutes of the drill. The finishing drill is flat-ended. If a guide wire is still in place at this point it may be removed, or it may be left in place to guide installation of the implant.

Alternately, a cannulated punch can be placed over the guide wire instead of the cannulated drill bit. The punch can be twisted or toggled so the material inside the punch breaks off near the end of the punch and can be removed with the punch.

In another embodiment, a drill sleeve may be placed over the guide wire before the cannulated drill bit. One or more drills can then be used to drill out the material in the drill sleeve.

In this embodiment, the guide wire may be a Kirschner wire (K-wire), which is a metal pin. The K-wire can be 1.5-2.0 mm in diameter. The cannulated drill bit is sized to fit over the guide wire and can be stepped so that the diameter of the shank of the drill bit is less than that of the cutting portion. The shank of the cannulated drill bit is sized to fit within the drill sleeve such that the drill sleeve guides the drill bit. The finishing drill bit is also sized to fit within the drill sleeve.

In addition, defects suitable for use with the implants of the invention can be created by any defect creation methods known to the art in combination with the special alignment tools and procedures described above for use with complex surfaces. In some situations, the surface shape around the defect may allow defect creation without using a specially shaped drill guide or a guide wire.

Cylindrical defects can be created by using a drill sleeve with a circular cross-section. Other shapes of defects can be created by using a drill sleeve with a non-circular cross-section. For example, a drill sleeve with a square cross-section can be used to create a defect in the form of a prism.

After the defect is created, the delivery devices of the invention can be used to insert the implant in the device. When the defect creation method leaves a guide wire in the center of the defect, a delivery device is used with a central hole in the inner shaft, the hole being sized to permit passage of the guide wire. Also, an implant with a central hole to permit passage of the guide wire can be used under these circumstances.

In an embodiment, the invention provides a method for delivering an implant plug having a nonplanar proximal surface into a defect in a tissue, the defect having an unmeasured depth, using the implant delivery device of the invention, the method comprising the steps of:
inserting said implant into the distal end of said loading device such that at least a portion of the proximal surface of the implant contacts the distal end of the inner shaft, wherein when said implant is disposed in said loading device the proximal end of the inner shaft protrudes from the proximal end of the outer shaft and the length of said implant equals the length of the protruding section of the inner shaft;
inserting the proximal end of the inner shaft into the defect until the proximal end of the inner shaft contacts the bottom of the defect;
advancing the outer shaft in the proximal direction until the proximal end of the outer shaft effectively conforms to the surface of the tissue surrounding the defect, causing a portion of the implant to extend beyond the distal end of the outer shaft;
cutting off the portion of the implant extending beyond the distal end of the outer shaft, leaving a remaining portion disposed within the outer shaft;
placing the distal end of the loading device over the defect to effectively conform to the tissue surrounding the defect; and
distally advancing the inner shaft to push the portion of the implant remaining after cutting into the defect.

The invention also provides kits for preparing a defect at a specified location in a tissue having a nonplanar surface. In an embodiment, the kit comprises:
a drill guide having a proximal end shaped to conform to the surface of the tissue at the perimeter of the defect;
a drill sleeve; and
a drill bit.

The implants and delivery devices of the invention can be employed with a capsule loader for loading the implant into the delivery device as described in U.S. patent application Ser. No. 10/785,388, filed Feb. 23, 2004. The implants and delivery devices of the invention can also be employed with cutting devices for trimming excess implant material from the distal end of the implant as described in U.S. patent application Ser. No. 10/785,388, filed Feb. 23, 2004.

All patents and publications mentioned in the specification are indicative of the levels of skill of those skilled in the art to which the invention pertains.

One skilled in the art would readily appreciate that the present invention is well adapted to carry out the objects and obtain the ends and advantages mentioned, as well as those inherent therein. The devices, methods and accessory methods described herein as presently representative of preferred embodiments are exemplary and are not intended as limitations on the scope of the invention. Changes therein and other uses will occur to those skilled in the art, which are encompassed within the spirit of the invention, are defined by the scope of the claims.

Although the description herein contains many specificities, these should not be construed as limiting the scope of the invention, but as merely providing illustrations of some of the embodiments of the invention. Thus, additional embodiments are within the scope of the invention and within the following claims. All references cited herein are hereby incorporated by reference to the extent that there is no inconsistency with the disclosure of this specification. Some references provided herein are incorporated by reference herein to provide details concerning additional starting materials, additional methods of synthesis, additional methods of analysis and additional uses of the invention.

When a Markush group or other grouping is used herein, all individual members of the group and all combinations and subcombinations possible of the group are intended to be individually included in the disclosure.

### EXAMPLE

Dual-Phase Implant Plug

The dual-phase implant plug has a proximal layer designed to have properties similar to that of cartilage and a distal layer designed to have properties similar to that of bone. Table 3 lists an exemplary composition for the bone phase, while Table 4 lists an exemplary composition for the cartilage phase. The PGA fibers listed in the tables are of poly-glycolic acid. Table 5 lists exemplary physical properties of bone and cartilage phases having the compositions listed in Tables 3 and 4.

TABLE 3
Bone phase:

| Component | Quantity (vol %) |
|---|---|
| Poly-lactic acid | 54% |
| PGA Fibers | 10% |
| Calcium Phosphate | 35% |
| Surfactant | 1% |

TABLE 4
Cartilage Phase

| Component | Quantity (vol %) |
|---|---|
| Poly-lactic-co- | 93% |
| glycolide, 75/25 | |
| PGA Fibers | 6% |
| Surfactant | 1% |

TABLE 5
Physical Properties

| | Bone Phase | | Cartilage Phase | |
|---|---|---|---|---|
| Porosity | 70% | | 80% | |
| Pore size | 100-600 | μm | 80-250 | μm |
| Strength | 25 | MPa | 1.5 | MPa |
| Stiffness | 150 | MPa | 25 | MPa |
| Phase thickness | 12.5 | mm | 2.5 | mm |

1. An implant plug for insertion into a defect in a tissue, the implant plug comprising a complex, clinically acceptable proximal surface.

2. The implant of claim 1, wherein the proximal surface of the implant comprises two facets converging to form an angled surface.

3. The implant of claim 2, wherein the angle between the facets is between about 70 and about 130 degrees.

4. The implant of claim 3, wherein the angle between the facets is between about 90 and about 110 degrees.

5. The implant of claim 4, wherein the angle between the facets is about 100 degrees.

6. The implant of claim 1 wherein the proximal surface of the implant is concave.

7. The implant of claim 1 wherein the proximal surface of the implant is convex.

8. The implant of claim 1 wherein the proximal surface of the implant is a multifaceted dome.

9. The implant of claim 1 wherein the proximal surface of the implant is saddle-shaped.

10. The implant of claim 1 wherein the proximal surface of the implant is beveled so that part of the proximal surface meets the side of the implant at an angle less than 90 degrees.

11. The implant of claim 1 which is a single phase plug.

12. The implant of claim 1 which is a multi-phase plug.

13. The implant of claim 12 which is a dual phase plug.

14. The implant of claim 1 wherein the implant is loaded with a bioactive agent.

15. The implant of claim 1, wherein the implant comprises a composite material comprising an absorbable polymer and a ceramic or mineral.

16. The implant of claim 15, wherein the composite material further comprises fibers.

17. A kit for inserting an implant plug having a complex proximal surface into a defect in a tissue comprising at least one implant of claim 1 and at least one implant delivery device comprising:
a tubular outer shaft having a proximal and distal end, a longitudinal axis, and an internal bore along the longitudinal axis of said outer shaft;
an inner shaft having a distal end and a proximal end suitable for insertion into a defect, said inner shaft adapted to fit within said internal bore of the outer shaft so that the inner shaft and the outer shaft are slidably engaged;
wherein the proximal and distal ends of the outer shaft conform to the surface of the tissue at the perimeter of the defect, and the distal end of the inner shaft conforms to the proximal surface of the implant and the implant is sized for use with the delivery device.

18. The kit of claim 17, further comprising a cutting device.

19. The kit of claim 17, comprising a plurality of tissue implant delivery devices, each having different sizes of internal bores and inner shafts.

20. The kit of claim 19, further comprising a plurality of implants, each implant being sized for use with at least one delivery device.

21. A kit for preparing a defect at a specified location in a tissue having a nonplanar surface, the kit comprising:
a drill guide having a proximal end shaped to conform to the surface of the tissue at the perimeter of the defect;
a drill sleeve; and
a drill bit

22. The kit of claim 21 wherein the drill sleeve has a circular cross-section.

23. The kit of claim 21, further comprising a finishing drill bit having a flat end.

24. A method for creation of a defect having a selected location, diameter and depth in tissue having a complex surface, the method comprising the steps of:
a. placing a drill guide so that the proximal end of the guide is in contact with the tissue at the perimeter of the selected defect location, wherein the proximal end of the drill guide is shaped to conform to the shape of the tissue at the perimeter of the selected defect location;
b. inserting a drill sleeve into the interior of the drill guide;
c. seating the drill sleeve into the tissue to the selected defect depth;
d. inserting a drill bit into the drill sleeve; and
e. drilling the drill bit to the selected defect depth.

25. A method for creation of a defect having a selected location, diameter and depth in tissue having a complex surface, the method comprising the steps of:
a. placing a guide wire central to the selected defect location and at a selected angle to the tissue surface around the selected defect location;
b. placing a first cannulated drill bit over the guide wire and drilling to the selected defect depth;
c. removing the first cannulated drill;
d. placing a second cannulated drill bit over the guide wire and drilling to the selected defect depth, the second cannulated drill bit having a flat end; and
e. removing the second cannulated drill bit and the guide wire.
